SEQUENCE LISTING (9 sequences)

SEQ ID NO: 1 - protein, 725 aa, Artificial Sequence
  rLZ-8-HSA sequence
SDTALIFRLAWDVKKLSFDYTPNWGRGNPNNFIDTVTFPKVLTDKAYTYRVAVSGRNLGVKPSYAVESDGSGKVNFLEYNSGYGIADTNTIGVFVVDPDTNNDFIIAGWNGGGGSSMKWVTFISLLFLFSSAYSRGVFRRDAHKSEVAHRFKDLGEENFKALVLIAFAGYLGGCPFEDHVKLVNEVTEFAKTCVADESAENCDKSLHTLFGDKLCTVATLRETYGEMADCCAKGEPERNECFLGHKDDNPNLPRLVRPEVDVMCTAFHDNEETFLKKYLYEIARRHPYFYAPELLFFAKRYKAAFTECCGAADKAACLLPKLDELRDEGKASSAKGRLKCASLGKFGERAFKAWAVARLSGRFPKAEFAEVSKLVTDLTKVHTECCHGDLLECADDRADLAKYICENGDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPGNLIKGNCELFEGLGEYKFGNALLVRYTKKVPGVSTPTLVEVSRNLGKVGSKCCKHPEAKRMPCAEDYLSVVLNGLCVLHEKTPVSDRVTKCCTESLVNRRPCFSALEVDETYVPKEFNAETFTFHADICTLSEKERGIKKGTALVELVKHKPKATKEGLKAVMDDFAAFVEKCCKADDKETCFAEEGKKLVAASGAALGL

SEQ ID NO: 2 - protein, 350 aa, Artificial Sequence
  rLZ-8-Fc1 sequence
RPSDTALIFRLAWDVKKLSFDYTPNWGRGNPNNFIDTVTFPKVLTDKAYTYRVAVSGRNLGVKPSYAVESDGSGKVNFLEYNSGYGIADTNTIGVFVVDPDTNNDFIIAGWNGGGGSSEPKSCDKTHTCPPCPAPELLGGPSVFLFPPKPKDTLMISRTPEVTCVVVDVSHEDPEVKFNWYVDGVEVHNAKTKPREEGYNSTYRVVSVLTVLHGDWLNGKEYKCKVSNKALPAPIEKTISKAKGGPREPGVYTLPPSREEMTKNGVSLTCLVKGFYPSDIAVEWESNGGPENNYKTTPPVLDSDGSFFLYSKLTVDKSRWGGGNVFSCSVMHEALHNHYTGKSLSLSPGK

SEQ ID NO: 3 - protein, 507 aa, Artificial Sequence
  ACETYLATION (239), (375), (385), (398), (400), (476): rLZ-8-Fc4 sequence
RPSDTALIFRLAWDVKKLSFDYTPNWGRGNPNNFIDTVTFPKVLTDKAYTYRVAVSGRNLGVKPSYAVESDGSGKVNFLEYNSGYGIADTNTIGVFVVDPDTNNDFIIAGWNGGGGSSYTGRFKDKAKLTAVTSANTAYMELSSLTNEDSAVYYCSIIYFDYADFIMDYWGGGTTVTVSTASTKGPSVFPLAPCSRSTSESTAALGCLVKDYFPEPVTVSWNSGALTSGVHTFPAVLGXSGLYSLSSVVTVPSSSLGTKTYTCNVDHKPSNTKVDKRVESKYGPPCPSCPAPEFLGGPSVFLFPPKPKDTLMISRTPEVTCVVVDVSGEDPEVGFNWYVDGVEVHNAKTKPREEGFNSTYRVVSVLTVLHGDWLXGKEYKCKVSXKGLPSSIEKTISXAXGGPREPGVYTLPPSGEEMTKNGVSLTCLVKGFYPSDIAVEWESNGGPENNYKTTPPVLDSDGSFFLYSRLTVDKSXWGEGNVFSCSVMHEALHNHYTGKSLSLSLGK

SEQ ID NO: 4 - DNA, 25 bp, Artificial Sequence
  RT-PCR forward primer
cataggccttctgatactgctttga

SEQ ID NO: 5 - DNA, 23 bp, Artificial Sequence
  RT-PCR reverse primer
cggggtaccgaattcctattaca

SEQ ID NO: 6 - DNA, 25 bp, Artificial Sequence
  RT-PCR forward primer
gttaggccttctgatactgctttga

SEQ ID NO: 7 - DNA, 23 bp, Artificial Sequence
  RT-PCR reverse primer
tagggtacctcatttaccagggg

SEQ ID NO: 8 - DNA, 22 bp, Artificial Sequence
  RT-PCR forward primer
ccgaggccttctgatactgctt

SEQ ID NO: 9 - DNA, 23 bp, Artificial Sequence
  RT-PCR reverse primer
gatggtacctcacggagcatgag

(19) United States
(12) Patent Application Publication
(10) Pub. No.: US 2016/0289280 A1
(43) Pub. Date: Oct. 6, 2016

(54) Fusion protein of recombinant ganoderma immunoregulatory protein and human serum albumin, preparation method thereof, and application thereof

(71) Applicants: Xitian ZHANG, Shanghai (CN); Fei SUN, Shanghai (CN)

(72) Inventors: Xitian Zhang, Shanghai (CN); Fei Sun, Shanghai (CN); Chongyang Liang, Shanghai (CN)

(21) Appl. No.: 14/914,258

(22) PCT Filed: Jul. 11, 2014
(86) PCT No.: PCT/CN2014/082031

§ 371 Date: Feb. 25, 2016

(30) Foreign Application Priority Data
Oct. 15, 2013 (CN) 201310479016.0

(51) Int. Cl. C07K 14/375; C07K 14/765

(52) U.S. Cl. CPC C07K 14/375; C07K 14/765; C07K 2319/31; A61K 38/00

(57) ABSTRACT

The present invention relates to a field of biological pharmacy, wherein a fusion protein rLZ-8 of a ganoderma immunoregulatory protein and HSA prepared with gene engineering technologies, a preparation method thereof and applications thereof are disclosed. Compared with rLZ-8, a half-life of the fusion protein is prolonged, and biological activity thereof is increased. The fusion protein is applicable to drugs in treatment of leucopenia and anti-tumor.

### CROSS REFERENCE OF RELATED APPLICATION

This is a U.S. National Stage under 35 U.S.C 371 of the International Application PCT/CN2014/082031, filed Jul. 11, 2014, which claims priority under 35 U.S.C. 119(a-d) to CN 201310479016.0, filed Oct. 15, 2013.

### BACKGROUND OF THE PRESENT INVENTION

1. Field of Invention

The present invention relates to construction, expression, purification and application of a recombinant fusion protein, and more particularly to a preparation method and an application of a fusion protein of a recombinant ganoderma immunoregulatory protein and a human serum albumin, which is used for cure diseases such as leukopenia and tumor caused by chemotherapy.

2. Description of Related Arts

Ganoderma immunoregulatory protein (LZ-8) comes from a mycelium of ganoderma tsugae. A structure of the recombinant ganoderma immunoregulatory protein comprises: an important N-terminal domain for forming a dimer and a C-terminal FNIII domain; wherein the N-terminal domain of the rLZ-8 comprises an α-helix and a β-strand, an α-helix and a β-strand of an N-terminal of an LZ-8 monomer form an important dumbbell-shaped dimer binding domain through space exchanging with the respective domains of another LZ-8. It has been reported that the LZ-8 has biological activity on immunoloregulation (referring to Chinese patent CN201110222012.5) and killing tumor cells (referring to Chinese patent ZL200810050206.X).

Conventionally, main reasons for a short half-life of protein drugs are: 1) hydrolysis is one of the metabolism methods for most proteins, and existence of protein enzymes in body tissues reduces the activity of the protein; 2) kidney is the most important organ in the process of breakdown and metabolism of small-molecular protein; most proteins, with relative molecular weight of less than 69000 Da, are able to be excreted by glomerulus filtration. 3) liver plays an important role in the process of protein drug metabolism, wherein drug is delivered to liver cells through diffusion and carrier transport, and then is degraded by microsomal enzyme P450, protease or lysosome in cytosol; peptides and proteins with larger molecules are absorbed by the liver cells through endocytosis mediated by a receptor before being degraded by liver cells. A molecular weight of the dimer is less than 26 kDa; a clearance rate may be high; and pharmacokinetic parameters are difficult to meet requirements of pharmaceutical developments. Therefore, only by extending duration of the LZ-8 in vivo by gene-level fusion and other technical methods can solid foundations be laid for clinical application. In order to prolong the half-life of the protein drugs, in recent years, researchers mainly studies from aspects such as albumin fusion, chemical modification, microencapsulation, construction of mutants, and glycosylation. As the research progresses, new varieties of long-acting protein drugs are emerging.

Gene fusion technology connects different genes, for expressing a fusion protein with complex functions. Through gene fusion technology, molecular weights of the peptide and protein drugs are increased or affinity between the drug and the receptor is changed, so as to extend the half-life of drugs. Principle of constructing the fusion protein are as follows: removing a stop codon of a coding gene of a protein, and then connecting a coding gene of another protein with a stop codon, so as to realize fusion of the coding genes of the two proteins, for simultaneously expressing two proteins. The fusion protein gene has high stability and is able to be regulated and expressed, product thereof is homogeneous, affects on activity of protein and peptide drugs is small, etc., which conventionally provide a sufficient method for studying long-acting peptide and protein drugs.

Conventionally, widely studied fusion genes are human serum albumin gene, human immunoglobulin (IgG4, IgG1) gene, etc. Human serum albumin (HSA for short) is a protein in human plasma, non-glycosylated single-chain polypeptide thereof comprises 585 amino acids, and a molecular weight is 66 kDa. A concentration of HSA in plasma is 42 g/L, which is about 60% of total plasma proteins. Human serum albumin in the body fluids is able to transport fatty acids, bile pigments, amino acids, steroid hormones, metal ions, different therapeutic molecules, etc; while normal blood pressure is maintained. Clinically, human serum albumin is applicable to treatment of shock and burn, and is able to be used as a supplement to blood loss caused by surgery, accident or bleeding; human serum albumin is also able to be used as plasma compatibilizer.

Human immunoglobulin (IgG) is the most abundant protein in human blood, whose half-life is 21 days. It has been reported that a fusion of an Fc fragment of IgG and other proteins is able to significantly increase the biological activity and half life of other proteins in vivo, wherein most researchers choose IgG1 and IgG4 Fc fragments as a fusion object. The method has been widely used in some clinically important cell factors and soluble receptors, such as sTNF-αR, LFA3, CTLA-4, IL-2, and IFN-α, with significant success.

In view of the above background technology, the present invention uses the gene recombination technology to respectively fuse ganoderma immunoregulatory protein with human serum albumin and Fc fragment of human immunoglobulin IgG, so as to construct a eukaryotic expression system. After expression and purification, a target protein is obtained, and related biological activity and pharmaceutical researches are provided to the target protein. Result shows that the ganoderma immunoregulatory protein (LZ-8) is significantly different from the HSA fusion protein in pharmaceutical and biological activities as well as pathological application.

### SUMMARY OF THE PRESENT INVENTION

An object of the present invention is to provide a fusion protein of a recombinant ganoderma immunoregulatory protein and a human serum albumin, and a preparation method thereof, in such a manner that applications thereof in curing diseases such as leukopenia caused by chemotherapy and anti-tumor drugs are studied.

A fusion protein sequence: according to the present invention, the fusion protein rLZ-8-HSA of a ganoderma immunoregulatory protein (LZ-8) and an HSA comprises the ganoderma immunoregulatory protein and the HSA; an amino acid sequence thereof is:

(SEQ ID NO. 1)
SDTALIFRLAWDVKKLSFDYTPNWGRGNPNNFIDTVTFPKVLTDKAYTYR
VAVSGRNLGVKPSYAVESDGSQKVNFLEYNSGYGIADTNTIQVFVVDPDT
NNDFIIAQWNGGGGSSMKWVTFISLLFLFSSAYSRGVFRRDAHKSEVAHR
FKDLGEENFKALVLIAFAQYLQQCPFEDHVKLVNEVTEFAKTCVADESAE
NCDKSLHTLFGDKLCTVATLRETYGEMADCCAKQEPERNECFLQHKDDNP
NLPRLVRPEVDVMCTAFHDNEETFLKKYLYEIARRHPYFYAPELLFFAKR
YKAAFTECCQAADKAACLLPKLDELRDEGKASSAKQRLKCASLQKFGERA
FKAWAVARLSQRFPKAEFAEVSKLVTDLTKVHTECCHGDLLECADDRADL
AKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKC
CAAADPHECYAKVFDEFKPLVEEPQNLIKQNCELFEQLGEYKFQNALLVR
YTKKVPQVSTPTLVEVSRNLGKVGSKCCKHPEAKRMPCAEDYLSVVLNQL
CVLHEKTPVSDRVTKCCTESLVNRRPCFSALEVDETYVPKEFNAETFTFH
ADICTLSEKERQIKKQTALVELVKHKPKATKEQLKAVMDDFAAFVEKCCK
ADDKETCFAEEGKKLVAASQAALGL,

wherein an amino acid sequence of a connecting peptide connecting a C-terminal of the ganoderma immunoregulatory protein and the human serum albumin is GGGGSS.

According to the present invention, a fusion protein rLZ-8-Fc1 of the ganoderma immunoregulatory protein (LZ-8) and a human IgG1Fc fragment comprises the ganoderma immunoregulatory protein and the IgG1Fc fragment; wherein an amino acid sequence thereof is:

(SEQ ID NO. 2)
RPSDTALIFRLAWDVKKLSFDYTPNWGRGNPNNFIDTVTFPKVLTDKAY
TYRVAVSGRNLGVKPSYAVESDGSQKVNFLEYNSGYGIADTNTIQVFVVD
PDTNNDFIIAQWNGGGGSSEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K,

wherein the amino acid sequence of the connecting peptide connecting the C-terminal of the ganoderma immunoregulatory protein and the human serum albumin is GGGGSS.

According to the present invention, a fusion protein rLZ-8-Fc4 of the ganoderma immunoregulatory protein (LZ-8) and a human IgG4Fc fragment comprises the ganoderma immunoregulatory protein and the IgG4Fc fragment; wherein an amino acid sequence thereof is:

(SEQ ID NO. 3)
RPSDTALIFRLAWDVKKLSFDYTPNWGRGNPNNFIDTVTFPKVLTDKAY
TYRVAVSGRNLGVKPSYAVESDGSQKVNFLEYNSGYGIADTNTIQVFVVD
PDTNNDFIIAQWNGGGGSSYTQRFKDKAKLTAVTSANTAYMELSSLTNED
SAVYYCSIIYFDYADFIMDYWGQGTTVTVSTASTKGPSVFPLAPCSRSTS
ESTAALGCLVKDYFPEPVTVSWNSGALTSGVHTFPAVLQXSGLYSLSSVV
TVPSSSLGTKTYTCNVDHKPSNTKVDKRVESKYGPPCPSCPAPEFLGGPS
VFLFPPKPKDTLMISRTPEVTCVVVDVSQEDPEVQFNWYVDGVEVHNAKT
KPREEQFNSTYRVVSVLTVLHQDWLXGKEYKCKVSXKGLPSSIEKTISXA
XGQPREPQVYTLPPSQEEMTKNQVSLTCLVKGFYPSDIAVEWESNGQPEN
NYKTTPPVLDSDGSFFLYSRLTVDKSXWQEGNVFSCSVMHEALHNHYTQK
SLSLSLGK,

wherein the amino acid sequence of the connecting peptide connecting the C-terminal of the ganoderma immunoregulatory protein and the human serum albumin is GGGGSS.

Construction of engineering strains, as well as expression and purification of a target protein are as follows: respectively establishing carriers of nucleotide sequences of an LZ-8 fusion protein; using electricity transformation technology to obtain a host cell with Pichia pastoris as a fusion protein extracellular expression; wherein fermentation conditions of Pichia pastoris are optimized and expression value of the fusion protein is improved; purifying a target product by methods such as gravity column affinity chromatography, a molecular sieve, immobilized metal chelate affinity chromatography (IMAC) method, hydrophobic interaction chromatography (HIC), anion exchange chromatography, wherein purities of different fusion proteins obtained are above 99%, which provides crude drugs for subsequent pharmacological experiments.

Pharmacy experiments are as follows: using the different fusion proteins as contrasts of rLZ-8 for activity experiments, wherein through an ELISA method, a test result illustrates that a biological activity of the fusion protein rLZ-8-HSA is higher than a biological activity of the rLZ-8 which is not fused, and there is a significant difference, which is statistically significant; however, biological activities of the rLZ-8-Fc1 and the rLZ-8-Fc4 are lower than an activity index of the rLZ-8 which is not fused, and there is a significant difference; using the different fusion proteins as contrasts of the rLZ-8 for in vivo half-life experiments, wherein according to the ELISA method, in vivo half-lives of the different fusion proteins are all significantly improved to about 3 times of the one of the rLZ-8; meanwhile, using the different fusion proteins as contrasts of the rLZ-8 for leucopenia treating experiments, and counting white blood cells with an animal whole blood cell analyzer, wherein a test result illustrates that there is significant difference between the different fusion proteins and the rLZ-8 in treatment of the leukopenia; wherein with a same dosage, the fusion protein rLZ-8-HSA has a shortest cycle for promoting leukocyte growth; and with a same treatment cycle, the fusion protein rLZ-8-HSA promotes more white blood cells to grow. Therefore, the HSA is more suitable as a fusion partner of the rLZ-8, which is able to significantly enhance application of the LZ-8 in leukopenia treatment. According to melanoma cell growth inhibition experiments of the fusion protein rLZ-8-HSA, it is shown that with a same dosage (in LZ-8 meter), the fusion protein rLZ-8-HSA effectively inhibits melanoma cells growth, which is significantly different from a treatment effect of the rLZ-8 which is not fused. Meanwhile, according to a preferred embodiment of the fusion protein rLZ-8-HSA for inhibition of liver tumor cell growth, it can be seen that in a same treatment cycle, a cure rate of the fusion protein rLZ-8-HSA is significantly improved, which is unexpected to the inventor. According to the present invention, a preferred embodiment of the fusion protein rLZ-8-HSA for the treatment of thrombocytopenia is also provided. Accordingly, compared with a model group, an rLZ-8-HSA drug significantly stimulates proliferation of mouse platelets at a beginning of feeding, wherein a difference is extremely significant. In the mid-term of feeding, an effect thereof returns to a normal level. Sufficient effect is also observed in treatment of experimental animal models with thrombocytopenia caused by injection of anti-platelet serum.

Benefit effects of the present invention are as follows. According to the present invention, in vivo half-life of the fusion protein provided is significantly prolonged compared with the rLZ-8. Fusion protein constructed with gene fusion technologies will lower biological activity of a target protein after the fusion protein is produced. However, according to the present invention, the biological activity of the fusion protein rLZ-8-HSA has been experimentally proved to be significantly better than the biological activity of the rLZ-8 which is not fused. Besides, according to the present invention, fermentation of the fusion protein rLZ-8-HSA with the Pichia pastoris engineering strain is simple in technique, high in yield, single in expression product, and easy in purification, which provides favorable conditions for industrial production. There are problems such as an expression product is easy to be degraded during fermentation due to a Pichia pastoris expression system. Therefore, the present invention controls fermentation conditions, for greatly reducing degradation of the target products during Pichia pastoris fermentation expression, and increasing the yield. Through in vivo experiments, the present invention proves that compared with the rLZ-8 which is not fused, the in vivo half-life is significantly prolonged, while a lowest dosage and an onset time in the study of leukopenia treatment are also improved. Melanoma and liver tumor are researched as anti-tumor represents, and in vivo and in vitro experiments are respectively provided. According to the preferred embodiments of the melanoma and the liver tumor, therapeutic effects in the two experimental methods are significantly superior than the rLZ-8 not fused, which is unexpected to the inventor. According to the preferred embodiment of thrombocytopenia treatment, compared with the model group, the rLZ-8-HSA drug significantly stimulates proliferation of mouse platelets at the beginning of feeding, wherein the difference is extremely significant. In the mid-term of feeding, the effect thereof returns to the normal level, which is conventionally a best effect of the fusion protein of the rLZ-8.

### BRIEF DESCRIPTION OF THE DRAWINGS

FIG. 1 illustrates inducing expression of different fusion proteins at 66 h and 72 h;

wherein a lane 1 sample is a protein marker; a lane 2 sample is a standard HSA; a lane 3 sample is an rLZ-8; a lane 4 sample is a supernatant of an rLZ-8-HSA after being induced for 72 h; a lane 5 sample is a supernatant of the rLZ-8-HSA after being induced for 66 h; a lane 6 sample is a supernatant of an rLZ-8-Fc1 after being induced for 72 h; a lane 7 sample is a supernatant of the rLZ-8-Fc1 after being induced for 66 h; a lane 8 sample is a supernatant of an rLZ-8-Fc4 after being induced for 72 h; a lane 9 sample is a supernatant of the rLZ-8-Fc4 after being induced for 66 h.

FIG. 2 is a Western Bolt identification map of different fusion proteins;

wherein a lane 1 sample is a protein marker; a lane 2 sample is an rLZ-8; a lane 3 sample is an rLZ-8-HSA; a lane 4 sample is an rLZ-8-Fc1; a lane 5 sample is an rLZ-8-Fc4.

FIG. 3 is a chromatogram map of the fusion protein rLZ-8-HSA after being purified with a molecular sieve.

FIG. 4 illustrates in vivo half-life comparison of different fusion proteins and the rLZ-8.

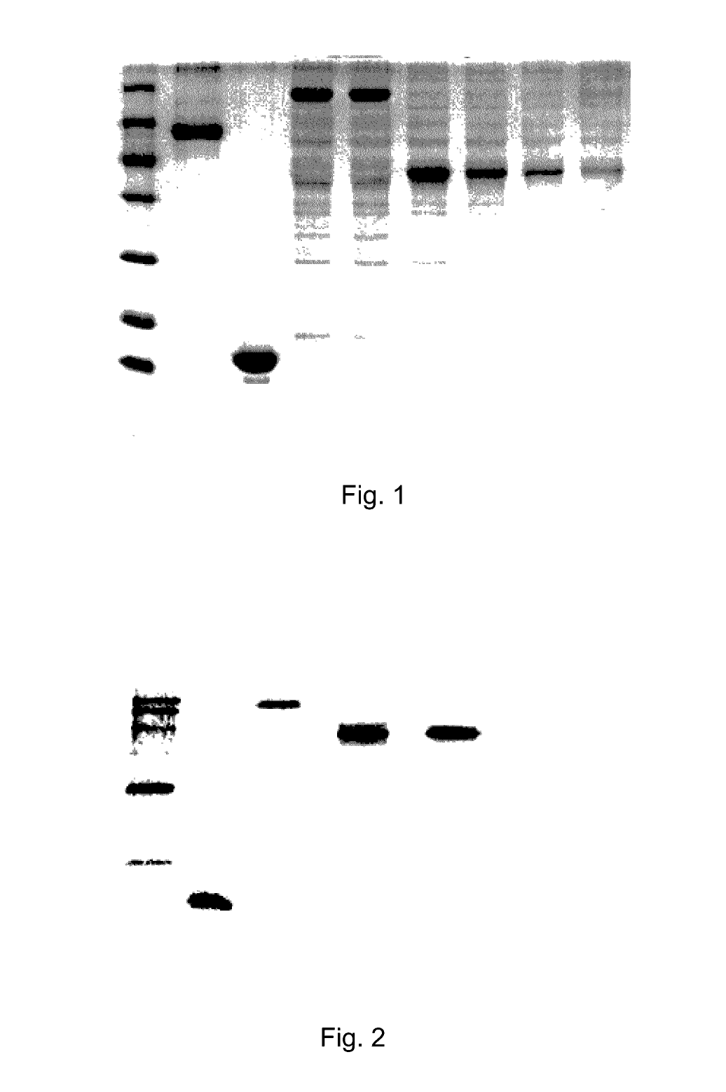

### DETAILED DESCRIPTION OF THE PREFERRED EMBODIMENT

### Preferred Embodiment 1

### rLZ-8 Fusion Protein Engineering Strains Construction and Expression

Construction: target fragments rLZ8-HSA, rLZ8-Fc1 and rLZ8-Fc4 sequences are respectively synthesized according to yeast codon preferences, and is stored in a puc57 plasmid; a primer comprising restriction enzyme cutting sites StuI and KpnI is design; the primer is synthesized as follows:

| | |
|---|---|
| (1) LZ-8-HSA: | 5′ CATAGGCCTTCTGATACTGCTTTGA 3′ |
| | 5′ CGGGGTACCGAATTCCTATTACA 3′ |
| (2) LZ-8-Fcl: | 5′ GTTAGGCCTTCTGATACTGCTTTGA 3′ |
| | 5′ TAGGGTACCTCATTTACCAGGGG 3′ |
| (3) LZ-8-Fc4: | 5′ CCGAGGCCTTCTGATACTGCTT 3′ |
| | 5′ GATGGTACCTCACGGAGCATGAG 3′ |

Obtaining the target fragments through PCR, wherein conditions for PCR are: firstly, 95° C. for 30 s; then 95° C. for 30 s, 58° C. for 30 s, and 72° C. for 2 min, wherein the above processes are repeated for 30 times; finally, 72° C. for 10 min, and standing by at 16° C.

Identifying by 1% agarose electrophoresis that the fragments are respectively at 2184 bp (LZ-8-HSA), 1064 bp (LZ-8-Fc1) and 774 bp (LZ-8-Fc4); treating a pPICZα A carrier and the target fragments according illustrations of a GENEART Seamless Cloning and Assembly Kit, wherein a mole ratio of the carrier and the fragments is 1:3, wherein the carrier and the fragments are reacted with 5× buffer and 10× enzyme mixture for 30 min; transforming 10 ul connection product into colibacillus competence DH5 alpha; after being cultured at 37° C. for 30 min, coating the connecting product on an LB plate with bleomycin resistance; selecting a single bacterial colony is selected to be cultured at 37° C. with a shaker for a night; centrifuging bacterial liquid with 12000 g, and removing supernatant; extracting a recombinant plasmid with a plasmid small extraction kit, and testing a sequence thereof; using double enzyme digestion and electrophoresis for identifying whether a converter is correct; finally, using 5′AOX and 3′AOX primers for testing correctness of the sequence.

Linearizing the recombinant plasmid with SacI enzyme at 37° C. for about 1 h; adding 33 times Pichia pastoris for inoculating to YPD at 30° C., and culturing with a 300 rpm shaker for a night; enlarging to 500 mL; when OD₆₀₀ reaches about 1.3, preparing yeast competence; ice-bathing for 30 min and re-suspending with ice bath sterile water; centrifuging with 1500 g at 4° C. for 5 min, and dividing into 80 ul yeast competence per tube; adding 10 ug linearizing plasmid; ice-bathing for 5 min; transferring to a electro-transforming device and providing electro-transforming under 1.5 kV, 500, 25 mA; shaker-culturing at 30° C. for 2 h, a coating on an YPDS plate with bleomycin Zeocin resistance, and culturing at 30° C. for 3 days.

screening: under an aseptic condition, selecting 20 mono-clones of each fusion protein on the YPDS plate with the bleomycin Zeocin resistance, and placing in 10 mL YPD liquid culturing base for culturing at 30° C. with a 300 rpm shake flask for 12 h; centrifuging with 1500 g for 5 min and removing supernatant; changing to a BMGY culturing base for culturing at 30° C. with a 300 rpm shake flask for 18 h; centrifuging with 1500 g for 5 min and removing supernatant; changing to a BMGY (with 1% methanol) culturing base, and adding 1% methanol once per 24 h; providing inducing expression for 72 h; centrifuging with 1500 g for 5 min, and keeping supernatant at −20° C.; quantitatively and qualitatively describing the expression of the target proteins through SDS electrophoresis and Western Bolt identification, so as to selecting high-expression engineering strains.

### Preferred Embodiment 2

### Purification Techniques of Fusion Proteins

Because the fusion protein has a public LZ-8 structure, purification methods such as gravity column affinity chromatography, molecular sieve, immobilized metal chelate affinity chromatography (IMAC) method, hydrophobic interaction chromatography (HIC), anion exchange chromatography are studied according to characteristics of the structure, wherein specific methods are as follows.

The purification methods of the rLZ-8-Fc1 and the rLZ-8-Fc4 are as follows.

Microfiltration: centrifuging a fermentation liquid with 10000 rpm for obtaining supernatant, then purifying (micro-filtering) by a hollow fiber column with a hole diameter of 100 Kd, so as to remove small molecular salts and sugars, for obtaining 8 L yellow clear liquid comprising pigment, nucleic acid and protein.

rProtein A Gravi Trap gravity column affinity chromatography: preparing buffer solution A phase: 0.22M phosphate buffer solution, pH 7.7, and 0.15M sodium chloride; preparing buffer solution B phase: 0.1M citrate buffer solution, pH 4.0, 0.22 μvacuum filtration, ultrasonic degassing; balancing a chromatographic column with the A phase phosphate buffer solution, wherein a volume thereof is 10 ml; respectively sampling 10 ml pre-treated rLZ-8-Fc1 and rLZ-8-Fc4 fermentation liquid, and combining for two times; (sample treatment: 90 ml the fermentation liquid and 10 ml 10× the phosphate buffer solution, 0.22 μfiltration and sterilized storage), washing the chromatographic column with the A phase phosphate buffer solution, eluting without combination; washing an eluting sample with the B phase citrate buffer solution, wherein a volume thereof is 10 ml; waiting for 2 min, and removing 1 ml volume in the chromatographic column; receiving the samples with a 1.5 ml centrifuging tube, and storing for testing; regenerating the chromatographic column after being used for 5 times, adding 5 ml 6M guanidine hydrochloride, waiting for 2 min, and washing the chromatographic column with the A phase.

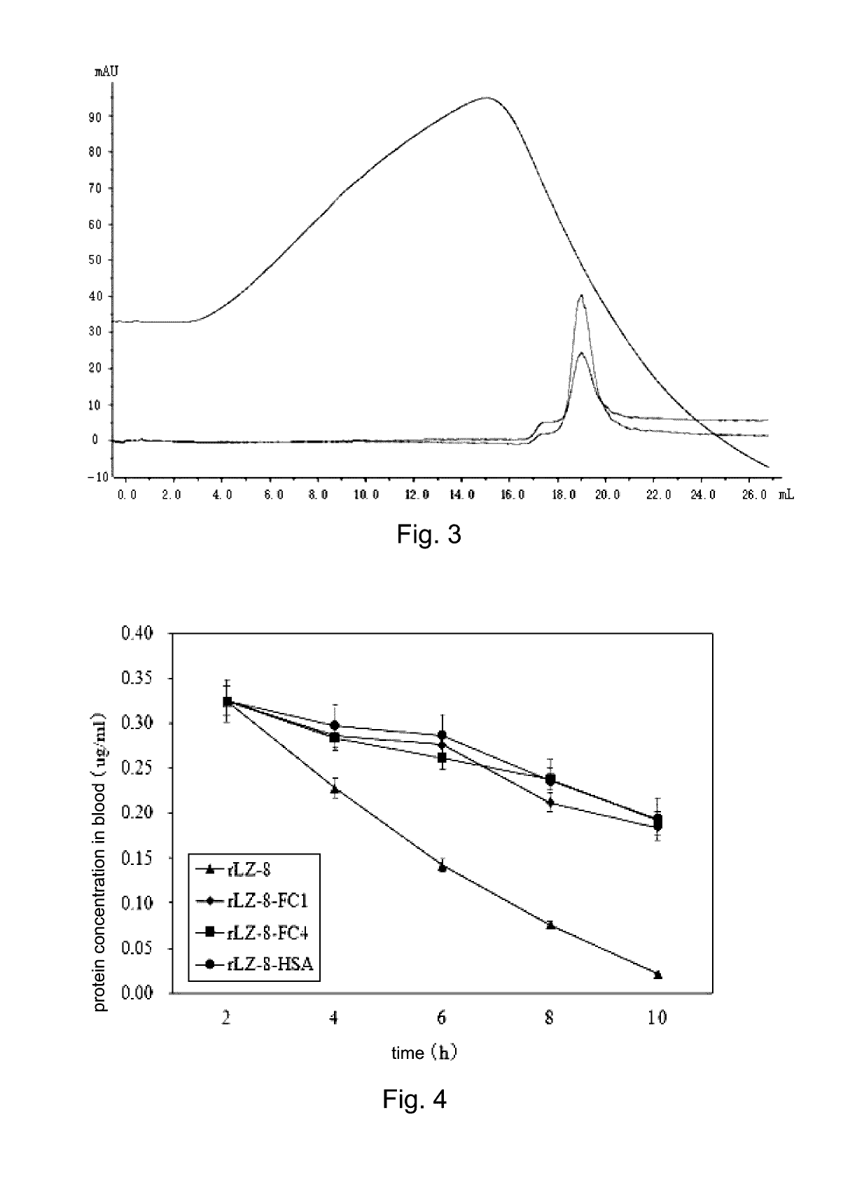

Molecular sieve chromatography: using a Superdex 75 filling column (GE, XK16/70, wherein an internal diameter thereof is 16 mm, a height thereof is 70 cm), wherein a filling height is 60 cm; testing with 100 μL 1% acetone, wherein a column efficiency is about 10000 according to a result; sampling protein with a flow rate of 2 mg/mL and a concentration of 5 mL, then eluting with pH 7.5, NaH₂PO₄—Na₂HPO₄ (50 mM) buffer solution (illustrated in FIG. 3); sampling at a collection peak, and providing electrophoresis and HPLC tests.

Results: after fine purification, a purity of the protein is above 99% according to the HPLC test, SDS-PAGE electrophoresis has a single strip.

A purification method of the rLZ-8-HSA comprises steps of:

step (1): purifying the rLZ-8-HSA with the Immobilized metal chelate affinity chromatography (IMAC) method, wherein a filler IMAC Sepharose 6Fast Flow 1 is purchased for GE; filling a XK50/30 column with a filling height of 15 cm; replacing a storage liquid with pure water; passing 0.1M copper sulfate solution through a chromatography column, wherein a volume of the copper sulfate equals to a column volume, and washing copper ions which are not adsorbed with purified water, wherein a volume of the purified water is four times of the column volume; then balancing the chromatography column with a buffer solution A: 20 mmol/L phosphate, 0.6 mol/L sodium chloride, and pH 7.3, wherein a supernatant comprising the human serum albumin is added into the phosphate and the sodium chloride for forming 20 mmol/L phosphate, 0.6 mol/L sodium chloride, and pH 7.3; then sampling with an AKTA™ Purify chromatography system with a flow rate of 50 ml/min; after sampling, washing with the buffer solution A until an absorption value reaches a basic point; eluting the target protein with a buffer solution B: 20 mmol/L phosphate, 0.6 mol/L sodium chloride, 0.3M iminazole and pH 7.5; collecting an eluting peak of the buffer solution B;

step (2): purifying with hydrophobic interaction chromatography (HIC), wherein the eluting peak of the buffer solution B collected in the step (1) is purified with hydrophobic interaction chromatography; filling a chromatography having a diameter of 5 cm with a phenyl hydrophobic chromatography medium Phenyl Sepharose™ 6 Fast Flow (high sub) (from GE), wherein a column height is 15 cm; wherein before utilization, the medium is balanced with a buffer solution C: 50 mmol/L phosphate, 0.5M sodium chloride, and pH 6.5, and a volume of the buffer solution C is 3 times of a column volume; adding deionized water into the buffer solution B comprising the target protein, which is obtained in the step (1), diluting the a concentration of 0.5M NaCl, and adjusting a pH value to 6.5 with phosphoric acid; sampling with the AKTA™ Purify chromatography system with a flow rate of 50 ml/min; after sampling, eluting with the buffer solution C: 50 mmol/L phosphate, 0.5M sodium chloride, and pH 6.5, wherein the volume of the buffer solution C is 2 times of the column volume; collecting a flow through peak and an eluting peak with the buffer solution C; eluting a combination portion with the hydrophobic chromatography column with the deionized water having a volume two times of the column volume, and discharging effluent liquid; adding collected liquid from the hydrophobic chromatography column into sodium tetraborate and calcium chloride solution with a final concentration of 0.1M, adjusting pH to 9.0 and treating for 0.5-24 h, then centrifuging with 10000 rpm for 20 min, collecting supernatant, and desalinating with a MILLIPORE 10K ultrafiltration membrane; and step (3): fining the sample with anion exchange chromatography, loading a Q Sepharose™ High Performance filler into a chromatography with a diameter of 2.6 cm and a height of 15 cm, wherein a filling volume is 80 ml; washing with the deionized water, wherein a volume thereof is two times of a column volume; then balancing with a buffer solution E (50 mMpb, pH 7.0) having a volume five times of the column volume; after sampling, washing with the buffer solution E, wherein the volume thereof is tow times for the column volume; then eluting with 0-0.5M NaCl with 10 times column volume gradient, and collecting a main peak.

Results: after fine purification, a purity of the protein is above 99% according to the HPLC test, SDS-PAGE electrophoresis has a single strip.

### Preferred Embodiment 3

### Comparison of Different Fusion Proteins on Promotion of Mouse Spleen Cell Proliferation

Testing effects of the fusion proteins on mouse spleen cell proliferation by a WST-1 method, which illustrates strength of biological activity, wherein according to the present invention, BALB/c female mice are used, whose weight is controlled at 20-22 g; executing the mice by stretching necks thereof, taking out spleens under a sterile condition, and putting into a plate with 5 ml DMEM comprising 10% calf serum; cracking the spleens with tweezers, filtering tissue suspension with gauze for removing tissue blocks, and preparing cell suspension of spleen cells; adding 100 μl the tissue suspension into 900 μl 2% glacial acetic acid for counting with a microscope; adjusting a cell concentration to 5×10⁶/ml with DMEM comprising 2% calf serum; respectively preparing the fusion proteins and the rLZ-8 with same molar concentration gradient, wherein there are 3 gradient concentrations, each concentration occupies 9 wells with 100 μl per well; adding tissue suspension with a concentration of 5×10⁶/ml with 100 μl per well; shaking for evenly mixing, and then putting into a 37° C., 5% CO₂ incubation device to incubate for 24 h; after incubation, adding WST-1 with 20 μl per well; putting into the 37° C., 5% CO₂ incubation device to incubate for 3 h, then testing OD₄₅₀ (with a BIO-RAD); wherein results are listed in table 1.

TABLE 1
Biological activity of different fusion proteins on promotion of cell
proliferation (x ± s n = 9)

| Concen- | Protein | | | |
|---|---|---|---|---|
| tration | rLZ-8 | rLZ-8-Fc1 | rLZ-8-Fc4 | rLZ-8-HSA |
| 5.0 × 10⁻⁹ | 0.739 ± 0.23 | 0.543 ± 0.26 | 0.452 ± 0.43 | 0.936 ± 0.31* |
| mol/L | | | | |
| 10.0 × 10⁻⁹ | 1.197 ± 0.31 | 0.937 ± 0.29 | 0.840 ± 0.27 | 1.680 ± 0.38* |
| mol/L | | | | |
| 15.0 × 10⁻⁹ | 1.567 ± 0.27 | 1.114 ± 0.31 | 0.929 ± 0.21 | 2.373 ± 0.35* |
| mol/L | | | | |
| Note: | | | | |
| comparison with rLZ-8, *p < 0.05 | | | | |

Referring to table 1, with increase of the protein concentration, an effect of the fusion proteins on spleen cell proliferation also increases. With the same protein concentration, it can be concluded from proliferation effect comparison of the different fusion protein and the rLZ-8 that the rLZ-8-HSA is better than the rLZ-8 in promotion of the spleen cell proliferation, and there is a significant difference, which is statistically significant. However, promotion effects of the rLZ-8-Fc1 and the rLZ-8-Fc4 on the mouse spleen cell proliferation is significantly decreased. According to experimental results, active spots are not affected by fusion of LZ-8 and the HSA, while after fusion of the LZ-8 and IgG-Fc1 or IgG-Fc4, the protein activity is decreased, which hinders LZ-8 activity.

### Preferred Embodiment 4

### Half-Life Tests of Different Fusion Proteins

Utilizing BALB/c mice weighing about 18-22 g in experiments, wherein each group comprises 10 mice; intravenously injecting 100 g/kg (judging from LZ-8 dosage) the fusion protein, respectively sampling blood after 2, 4, 6, 8 and 10 hours after injection, drawing a curve of medicine concentration per time (illustrated in FIG. 4) with the results obtained, wherein it is indicated by the experiment results that the half-life of the fusion protein is significantly extended (P<0.0001) according to the rLZ-8 which is not fused, a concentration thereof in blood is greatly prolonged, and the half-life of the rLZ-8 in the mouse is improved.

### Preferred Embodiment 5

### Research of Fusion Protein rLZ-8-HSA on Treatment of

Utilizing Wistar rats in the experiments, wherein 18 rats weighing about 100 g are utilized. A method for preparing reagents comprises steps of: dissolving the rLZ-8 in sterile saline, and diluting into 60 μg/kg, 30 μg/kg and 15 μg/kg dosage groups; dissolving the fusion protein rLZ-8-HSA in the sterile saline, and diluting into 60 μg/kg, 30 μg/kg and 15 μg/kg (judging from the LZ-8 dosage) dosage groups; diluting GenLei®Scimax® [recombinant human granulocyte colony-stimulating factor injection (rhG-CSF)], batch number: 20130403, 75 μg/vial, into 13.5 μg/ml and 0.1 ml per rat with the sterile saline; diluting cyclophosphamide (CP) injection, batch number 13020225, 200 mg/vial, into 20 mg/ml and 0.1 ml per rat with the sterile saline, or 20 mg/kg.

The experiment has an rLZ-8 low-dosage group, an rLZ-8 middle-dosage group, an rLZ-8 high-dosage group, an rLZ-8-HSA low-dosage group, an rLZ-8-HSA middle-dosage group, an rLZ-8-HSA high-dosage group, and a positive medicine control group (utilizing the GenLei®Scimax®). The rats of each the group are injected with the cyclophosphamide in tail vein for three days except that the sterile saline is given to the normal control group, the dosage is 20 mg/ml and 0.1 ml per rat. On the third day, the blood is sampled from the tail vein, and the leukocytes are counted by a cytoanalyzer. After successful modeling, the rats of each the group are respectively treated with the rLZ-8, the three kinds of the fusion proteins, or the positive medicine (the GenLei®Scimax®) with the corresponding dosage, and the equal sterile saline is given to the rats of the normal control and a CP group. The blood is sampled from the tail vein on the first, third and seventh treatment days, and the leukocytes are counted by the cytoanalyzer. Medicine efficacy is analyzed according to a number difference of the leukocyte between before and after the treatment.

TABLE 2
effects of rLZ-8 on rats models with leucopenia (n = 10)

| | leukocyte | on the first | on the third | on the seventh |
|---|---|---|---|---|
| group | number before | treatment day | treatment day | treatment day |
| normal control | 14.11 × 10⁹ · L⁻¹ | 14.36 × 10⁹ · L⁻¹ | 13.8 × 10⁹ · L⁻¹ | 12.13 × 10⁹ · L⁻¹ |
| CP control | 5.1 × 10⁹ · L⁻¹ | 5.3 × 10⁹ · L⁻¹ | 5.8 × 10⁹ · L⁻¹ | 9.27 × 10⁹ · L⁻¹ |
| GenLei ® | 4.55 × 10⁹ · L⁻¹ | 6.4 × 10⁹ · L⁻¹ | 11.83 × 10⁹ · L⁻¹* | 11.17 × 10⁹ · L⁻¹ |
| Scimax ® | | | | |
| rLZ-8 | 3.71 × 10⁹ · L⁻¹ | 4.6 × 10⁹ · L⁻¹ | 9.3 × 10⁹ · L⁻¹* | 11.2 × 10⁹ · L⁻¹ |
| (low-dosage) | | | | |
| rLZ-8 | 3.12 × 10⁹ · L⁻¹ | 5.1 × 10⁹ · L⁻¹ | 9.7 × 10⁹ · L⁻¹* | 12.78 × 10⁹ · L⁻¹ |
| (middle-dosage) | | | | |
| rLZ-8 | 4.09 × 10⁹ · L⁻¹ | 5.4 × 10⁹ · L⁻¹ | 9.6 × 10⁹ · L⁻¹* | 14.5 × 10⁹ · L⁻¹ |
| (high-dosage) | | | | |
| rLZ-8-HSA | 3.11 × 10⁹ · L⁻¹ | 8.5 × 10⁹ · L⁻¹* | 10.8 × 10⁹ · L⁻¹** | 13.2 × 10⁹ · L⁻¹ |
| (low-dosage) | | | | |
| rLZ-8-HSA | 2.89 × 10⁹ · L⁻¹ | 9.2 × 10⁹ · L⁻¹* | 11.98 × 10⁹ · L⁻¹** | 13.4 × 10⁹ · L⁻¹ |
| (middle-dosage) | | | | |
| rLZ-8-HSA | 3.33 × 10⁹ · L⁻¹ | 8.4 × 10⁹ · L⁻¹* | 14.4 × 10⁹ · L⁻¹** | 15.5 × 10⁹ · L⁻¹ |
| (high-dosage) | | | | |

Note:
comparison with CP control group, *p < 0.05, **p < 0.01

It is illustrated in table 2 that on the first treatment day, the leukocyte number of the rats of the rLZ-8-HSA groups is significantly increased in comparison with the rats of the CP group, and on the seventh treatment day, the leukocyte number basically approaches to a normal level. On the first treatment day, the leukocyte number of the rats of the rLZ-8-HSA groups is significantly increased in comparison with the rats of the GenLei®Scimax® control group, and on the seventh treatment day, the leukocyte number basically approaches to the normal level. It is emphasized that when compared with the rLZ-8 groups with the same dosage, the rLZ-8-HSA groups have a sufficient leukocyte proliferation effect from the first treatment day, wherein the leukocyte number is about 2 times more than the leukocyte number of the rLZ-8 groups; with a same treatment period, the leukocyte proliferation effect of the rLZ-8-HSA low-dosage group is superior to the leukocyte proliferation effect of the rLZ-8 high-dosage group and other rLZ-8 groups.

### Preferred Embodiment 6

### Inhibition Effect of Fusion Protein rLZ-8-HSA on Melanoma

In vitro experiment: testing the inhibition effect of the fusion protein rLZ-8-HSA on the melanoma through a WST-1 method, preparing tissue suspension of the melanoma; adding 100 μl the tissue suspension into 900 μl 2% glacial acetic acid and counting with a microscope; adjusting a tissue concentration to 2×10⁵/ml with DMEM comprising 2% calf serum; respectively preparing the fusion protein rLZ-8-HSA and the rLZ-8 with same molar concentration gradient, wherein there are 3 gradient concentrations, each concentration occupies 9 wells with 100 μl per well; adding tissue suspension with a concentration of 2×10⁵/ml with 100 μl per well; shaking for evenly mixing, and then putting into a 37° C., 5% CO₂ incubation device to incubate for 24 h; after incubation, adding WST-1 with 20 μl per well; putting into the 37° C., 5% CO₂ incubation device to incubate for 3 h, then testing OD₄₅₀ (with a BIO-RAD); wherein results are listed in table 3.

TABLE 3
inhibition effect of fusion protein rLZ-8-HSA on melanoma (x ± s, n = 9)

Protein

| Concentration | rLZ-8 | rLZ-8-HSA |
|---|---|---|
| 5.0 × 10⁻⁹ mol/L | 0.427 ± 0.44 | 0.411 ± 0.31* |
| 10.0 × 10⁻⁹ mol/L | 0.372 ± 0.21 | 0.212 ± 0.29* |
| 15.0 × 10⁻⁹ mol/L | 0.302 ± 0.24 | 0.118 ± 0.11** |

Note:
comparison with rLZ-8, *p < 0.05, **p < 0.05

In vivo experiment: firstly, establishing a mouse tumor mould, wherein mouse melanoma cells B16-F10 is cultured with the DMEM comprising 10% fetal calf serum at 37° C. in a CO₂ incubation device; slowly subcutaneously injecting 200 μl B16-F10 cell suspension (comprising 1×10⁷ cells) with a 1 ml injector at mouse dorsal-ventral skin, so as to establish a mouse transplanted tumor mould.

Group arrangement and treatment method: 24 h after injecting the tumor cells, injecting at tail veins for rLZ-8 groups (123 μg/kg, 246 μg/kg and 492 μg/kg), rLZ-8-HSA groups (123 μg/kg, 246 μg/kg and 492 μg/kg judging from the LZ-8), and a dacarbazine group (2.5 mg/kg) or a normal saline group, wherein the rLZ-8 and the rLZ-8-HSA are injected once a day, the dacarbazine is continually injected for 5 days, and then injected again after 3 weeks, wherein a treatment cycle comprises 28 days; during experiment, observing living states of the mouse, weighing once every seven days, and sampling tail vein blood of the mouse once every 2 weeks; separating tumor bodies at an ending of the experiment, weighing the tumor bodies and recording; calculating an inhibition rate of the treating medicines on in situ tumor growth according to a formula that a tumor inhibition rate=(an average tumor weight of the normal saline group−an average tumor weight of the treated group)/an average tumor weight of the normal saline group.

Referring to experimental results: after weighing the tumor bodies, the average tumor weight of each group is calculated; according to table 4, it is illustrated that after 28 days, a tumor weight of a rLZ-8-HSA high-dosage group is less than tumor weights of other groups; within rLZ-8 groups or rLZ-8-HSA groups, the more a concentration of the medicine is, the less the tumor weight will be. Difference between the rLZ-8-HSA groups and a negative control group is extremely significant. Difference between the rLZ-8 low-dosage group, middle-dosage group and the high-dosage group is also significant (n=10, P<0.05). Difference between the rLZ-8-HSA low-dosage group, middle-dosage group and the high-dosage group is also significant (n=10, P<0.05).

TABLE 4
effect of rLZ-8 on B16-F10 in situ tumor weight

| | feeding method | | |
|---|---|---|---|
| | continually feeding for 28 days | | |
| | animal | | tumor |
| group | quantity | tumor weight (g) | inhibition rate (%) |
| normal saline | 10 | 1.24 ± 0.53 | — |
| dacarbazine | 10 | 0.52 ± 0.12** | 62.57 ± 0.27 |
| rLZ-8 | 10 | 0.53 ± 0.21**^{##} | 56.72 ± 0.41 |
| low-dosage | | | |
| rLZ-8 | 10 | 0.43 ± 0.17**^{##} | 64.91 ± 0.77 |
| middle-dosage | | | |
| rLZ-8 | 10 | 0.25 ± 0.04** | 83.04 ± 0.51^{##} |
| high-dosage | | | |
| rLZ-8-HSA | 10 | 0.35 ± 0.11**^{##} | 71.77 ± 0.31^{##} |
| low-dosage | | | |
| rLZ-8-HSA | 10 | 0.27 ± 0.14**^{##} | 78.22 ± 0.47^{##} |
| middle-dosage | | | |
| rLZ-8-HSA | 10 | 0.13 ± 0.05** | 89.51 ± 0.88^{##} |
| high-dosage | | | |

Note:
comparison with control group, *P < 0.05, **P < 0.01, wherein the experiment is repeated for three times, total results are trends to be the same, and the table 4 is only the result of one experiment;
comparison with the dacarbazine group, ^{#}P < 0.05, ^{##}P < 0.01, wherein the experiment is repeated for three times, total results are trends to be the same, and the table 4 is only the result of one experiment.

### Preferred Embodiment 7

### Inhibition Effect of Fusion Protein rLZ-8-HSA on Mouse 5180 Ehrlich Ascites Tumor

In vitro experiment: testing the inhibition effect of the fusion protein rLZ-8-HSA on the mouse 5180 Ehrlich ascites tumor through the WST-1 method, preparing tissue suspension of the mouse S180 Ehrlich ascites tumor; adding 100 μl the tissue suspension into 900 μl 2% glacial acetic acid and counting with a microscope; adjusting a tissue concentration to 2×10⁵/ml with DMEM comprising 2% calf serum; respectively preparing the fusion protein rLZ-8-HSA and the rLZ-8 with same molar concentration gradient, wherein there are 3 gradient concentrations, each concentration occupies 9 wells with 100 μl per well; adding tissue suspension with a concentration of 2×10⁵/ml with 100 μl per well; shaking for evenly mixing, and then putting into a 37° C., 5% CO₂ incubation device to incubate for 24 h; after incubation, adding WST-1 with 20 μl per well; putting into the 37° C., 5% CO₂ incubation device to incubate for 3 h, then testing OD₄₅₀ (with a BIO-RAD); wherein results are listed in table 5.

TABLE 5
inhibition effect of fusion protein rLZ-8-HSA on mouse S180 Ehrlich
ascites tumor (x ± s, n = 9)

Protein

| Concentration | rLZ-8 | rLZ-8-HSA |
|---|---|---|
| 5.0 × 10⁻⁹ mol/L | 0.453 ± 0.23 | 0.408 ± 0.21* |
| 10.0 × 10⁻⁹ mol/L | 0.366 ± 0.31 | 0.233 ± 0.18* |
| 15.0 × 10⁻⁹ mol/L | 0.345 ± 0.27 | 0.127 ± 0.15** |

Note:
comparison with the rLZ-8 groups, *p < 0.05, **p < 0.05

In vivo experiment: experimental material: mice weighing 18-22 g from Laboratory Animal Center of Jilin University; Ehrlich ascites tumor cell strains are provided by a lab of the inventor; cytoxan (CTX) is from Jiangsu Hengrui Medicine Co., Ltd, whose batch number is 06101921; 5180 ascites tumor and soild tumor experimental groups respectively comprises a normal control group, a negative control group, a positive control group, an rLZ-8 low-dosage group (0.25 mg·kg⁻¹), an rLZ-8 middle-dosage group (0.5 mg·kg⁻¹), an rLZ-8 high-dosage group (1 mg·kg⁻¹), an rLZ-8-HSA low-dosage group (0.25 mg·kg⁻¹), an rLZ-8-HSA middle-dosage group (0.5 mg·kg⁻¹), and an rLZ-8-HSA high-dosage group (1 mg·kg⁻¹), wherein dosages of the rLZ-8-HSA groups are judged from LZ-8 dosage, wherein each group comprises 10 mice.

Experimental method: experimental method of subcutaneous inhibition of the S180 tumor: selecting well-grown 5180 cells, diluting by sterile saline with a proper amount for preparing tumor cell suspension, whose cells are counted as 10⁷ L⁻¹, subcutaneous injecting at a right armpit of each mouse with a dosage of 20 ml (except for the normal control group); after 24 h, treating the mice; abdominally injecting normal saline to the normal and negative control groups with a dosage of 0.2 ml per mouse per day; abdominally injecting 20 mg·kg⁻¹ cyclophosphamide to the positive control group with a dosage of 0.2 ml per mouse per day; providing tail vein injection to the rLZ-8 groups with corresponding dosages, 0.2 ml per mouse per day and lasting for 10 days; before injection and 10 d after injection, sampling blood from mouse orbital venous plexus, and counting white blood cells by a clinical laboratory of First Hospital of Jilin university; on a next day after injection, executing all the mice by cervical dislocation, disserting and taking out tumor blocks, weighing and calculating a tumor inhibition rate according a following formula:

tumor inhibition rate (%)=(average tumor weight of control groups−average tumor weight of experimental groups)/average tumor weight of control groups×100%.

Experimental result: experimental results of subcutaneous inhibition of the S180 tumor: referring to table 6, the three rLZ-8 groups are all able to inhibit S180 growth, wherein tumor inhibition rates thereof are respectively 16.8%, 25.7% and 45.5%. Compared with the negative control group, tumor weights of the rLZ-8 groups have significant difference (P<0.01). Compared with the negative control group, tumor weights of the rLZ-8-HSA groups have significant difference (P<0.01). Meanwhile, compared with the rLZ-8 groups, tumor weights of the rLZ-8-HSA groups also have significant difference (P<0.01).

TABLE 6
inhibition effect of rLZ-8 on mouse transplanted tumor S180
(x ± s, n = 10)

| Group | Tumor weight (g) | Inhibition rate (%) |
|---|---|---|
| negative control | 1.01 ± 0.03 | — |
| CTX | 0.36 ± 0.02* | 64.3 |
| rLZ-8 | 0.83 ± 0.03* | 17.8 |
| low-dosage | | |
| rLZ-8 | 0.74 ± 0.02* | 26.7 |
| middle-dosage | | |
| rLZ-8 | 0.57 ± 0.03* | 43.5 |
| high-dosage | | |
| rLZ-8-HSA | 0.73 ± 0.03*^{#} | 27.7 |
| low-dosage | | |
| rLZ-8-HSA | 0.44 ± 0.05*^{#} | 56.4 |
| middle-dosage | | |
| rLZ-8-HSA | 0.21 ± 0.08**^{##} | 29.3 |
| high-dosage | | |

Note:
comparison with the negative control group, *P < 0.05, **P < 0.01; comparison with the rLZ-8 groups, ^{#}P < 0.05, ^{##}P < 0.01.

### Preferred Embodiment 8

### Pharmacodynamics Experiment of Fusion Protein rLZ-8-HSA on Treatment of Mouse Thrombocytopenia Caused by Cyclophosphamide

Experimental medicine: preparing a 19.25 μg·kg⁻¹ and a 9.625 μg·kg⁻¹ dosage groups of the recombinant ganoderma immunoregulatory protein (rLZ-8) with sterile injection water, and 0.2 ml per mouse; and preparing a 19.25 μg·kg⁻¹ and a 9.625 μg·kg⁻¹ dosage groups (judging from the LZ-8) of the fusion protein rLZ-8-HSA of the recombinant ganoderma immunoregulatory protein.

Positive control medicine: thrombopoietin (THPO) from Shenyang 3SBio Inc., with a dosage of 770 μg·kg⁻¹/d and 0.2 ml per mouse.

Chemotherapy medicine: cyclophosphamide (Cy) from Jiangsu Hengrui Medicine Co., Ltd, whose batch number is 12112121, 200 mg/bottle; preparing into 100 mg·kg⁻¹, 0.2 ml per mouse with sterile injection water.

Platelet dilution: urea: 1.3 g; sodium citrate: 0.5 g; formaldehyde: 0.1 ml; adding distilled water to 100 ml for mixing, then filtering for further utilization.

Experimental method: dividing experimental animals into 5 groups, wherein each group comprises 10 mice with 5 males and 5 females; except for a normal control group (injected with normal saline having an equal volume), abdominally injecting the cyclophosphamide to the mice with a dosage of 100 mg·kg⁻¹, 0.2 ml per mouse per day, and lasting for three days; when a platelet quantity decreased to below 300×10⁹/L, subcutaneously injecting the rLZ-8, the fusion protein rLZ-8-HAS (19.25 μg·kg⁻¹, 9.625 μg·kg⁻¹, 0.2 ml·per mouse per day) and the positive medicine (THPO 770 μg·kg⁻¹, 0.2 ml·per mouse per day) according to the above groups with corresponding dosages, and injecting the normal saline having the equal volume to the normal control group and a CP group; respectively sampling mouse tail vein blood 3 d, 7 d and 14 d after treatment, counting platelets with high power lens, wherein results thereof are listed in table 7.

Experimental result: compared with the CP group, platelets of the mice of the treated groups significantly increased on the third day, and returned to normal at the seventh day, wherein significant difference exists (p<0.05); platelets of the mice of the rLZ-8-HSA groups significantly increased, and returned to normal at the third day, wherein significant difference exists (p<0.05) compared with the CP group, which is statistically significant.

TABLE 7
experimental results of rLZ-8 on treatment of mouse
thrombocytopenia (x ± s, n = 10) (unit: *10⁹/L)

| Group | before | after | 3^{rd} day | 7^{th} day | 14^{th} day |
|---|---|---|---|---|---|
| normal control | 838 ± 0.11 | 946 ± 0.18 | 870 ± 0.23 | 760 ± 0.31 | 965 ± 0.70 |
| CP | 850 ± 0.34 | 239 ± 0.12 | 256 ± 0.93 | 301 ± 0.30 | 323 ± 0.67 |
| THPO | 851 ± 0.34 | 284 ± 0.19 | 390 ± 0.38 | 420 ± 0.32 | 689 ± 0.65 |
| rLZ-8 | 839 ± 0.40 | 201 ± 0.41 | 483 ± 0.45 | 585 ± 0.78 | 898 ± 0.61* |
| (9.625 μg · kg⁻¹) | | | | | |
| rLZ-8 | 946 ± 0.45 | 226 ± 0.78 | 501 ± 0.48 | 680 ± 0.28 | 1021 ± 0.31** |
| (19.25 μg · kg⁻¹) | | | | | |
| rLZ-8-HSA | 843 ± 0.65 | 261 ± 0.52 | 624 ± 0.43* | 697 ± 0.43* | 929 ± 0.61** |
| (9.625 μg · kg⁻¹) | | | | | |
| rLZ-8-HSA | 852 ± 0.82 | 246 ± 0.36 | 760 ± 0.38* | 978 ± 0.67* | 1035 ± 0.78** |
| (19.25 μg · kg⁻¹) | | | | | |

Note:
there is significant different when compared with the CP group, wherein *p < 0.05, **p < 0.01

### Preferred Embodiment 9

### rLZ-8-HSA Anti-Tumor Composition Preparation

1) According to the above pharmacological experiments, it is proved that an anti-tumor effect of the rLZ-8-HSA is extremely effective on maintaining body white blood cell level without toxic side effect. Therefore, it is considered that the rLZ-8-HSA is suitable for medicine and is safe.

2) According to the present invention, application of the rLZ-8-HSA as an anti-tumor medicine is able to be an oral form and a parenteral form. A dosage thereof depends on factors such as symptoms, age, and weight. For adults with the oral form, 10-1000 mg a time and several times a day; for the parenteral form, 10-100 mg a time and several times a day.

3) The present invention comprises oral tablets and pill capsules (comprising hard and soft capsules), wherein the above forms comprises the rlz-8 and at least one inert diluent (e.g. lactose, mannitol, glucose, starch, polyvinyl pyrrolidone). Besides the inert diluent, pharmaceutically acceptable additives such as lubricants, disintegrating agent, and stabilizer are also able to be added. If necessary, tablets or pills may be coated with a gastric or enteric soluble material by one or more layers. Non-parenteral injection comprises the rLZ-8-HSA and at least one inert diluent (such as distilled water, saline solution). Or, the rLZ-8-HSA may be prepared into freeze-dried powders, which is dissolved in the inert diluent for injection before utilization.

(1) Preparation 1

Dissolving 1000 mg the rLZ-8-HSA in 100 ml sterile saline, evenly mixing, and dividing the rLZ-8-HSA into drug bottles with a concentration of 10 mg/ml/tube, sealing and sterilizing for forming a final product; wherein other events are in line with injection liquid requirements of Pharmacopoeia of the People's Republic of China, 2010.

(2) Preparation 2

Preparing capsules with 100 g the rLZ-8-HSA and 0.5 kg pharmaceutical starch with common capsule preparation technologies and devices, wherein the rLZ-8-HSA is divided into 10 mg/grain, wherein other events are in line with capsule requirements of Pharmacopoeia of the People's Republic of China, 2010.

(3) Preparation 3

Preparing pills with 100 g the rLZ-8-HSA, 560 g microcrystalline cellulose, 380 g lactis anhydrous and 200 g magnesium stearate with common pill preparation technologies and devices, wherein the rLZ-8-HSA is divided into 10 mg/pill, wherein other events are in line with pill requirements of Pharmacopoeia of the People's Republic of China, 2010.

(4) Preparation 4

Preparing oral liquid with a proper amount of the rLZ-8-HSA with common oral liquid preparation technologies and devices, which is in line with oral liquid requirements of Pharmacopoeia of the People's Republic of China, 2010.

What is claimed is:

1. A fusion protein (rLZ-8-HSA) of a recombinant ganoderma immunoregulatory protein and a human serum albumin, wherein a C-terminal of a ganoderma immunoregulatory protein is connected to the human serum albumin through a connecting peptide, and an amino acid sequence of the fusion protein is SEQ ID NO. 1.

2. A method for preparing a medicine for leukopenia caused by chemotherapy, comprising applying a fusion protein rLZ-8-HSA as recited in claim 1.

3. A method for preparing a medicine for melanoma and liver tumor, comprising applying a fusion protein rLZ-8-HSA as recited in claim 1.

4. A method for preparing a medicine for thrombocytopenia, comprising applying a fusion protein rLZ-8-HSA as recited in claim 1.

5. The medicine, as recited in claim 2, 3 or 4, comprising a fusion protein as recited in claim 1 and any pharmaceutically acceptable auxiliary agent.

6. The medicine, as recited in claim 5, wherein the medicine is fed in an oral form and a parenteral form; the oral form comprises oral liquid, tablets, pills and capsules; the parenteral form comprises topical medicines and injections.
